(19) United States
(12) Patent Application Publication
(10) Pub. No.: US 2008/0287377 A1
(43) Pub. Date: Nov. 20, 2008

(54) Composition Based on Triethyl Citrate for the Prevention of Enzymatic Hydrolysis of Triglycerides

(76) Inventor: Gianfranco de Paoli Ambrosi, Salo (Brescia) (IT)

Correspondence Address: MCGLEW & TUTTLE, PC, P.O. BOX 9227, SCARBOROUGH STATION, SCARBOROUGH, NY, 10510-9227, US

(21) Appl. No.: 12/095,795

(22) PCT Filed: Nov. 29, 2006
(86) PCT No.: PCT/IT06/00827

§ 371 Date: Jun. 2, 2008

(30) Foreign Application Priority Data
Dec. 6, 2005 (IT) BS2005A000154

(51) Int. Cl. A61K 31/7048; A61K 31/194; A61K 31/201; A61K 31/351; A61P 17/00; A61K 31/4164; A61K 31/4025; A61K 31/203

(52) U.S. Cl. 514/29; 514/574; 514/560; 514/168; 514/422; 514/460; 514/398

(57) ABSTRACT

The invention concerns a composition for topical use for the protection of triglycerides against enzymatic hydrolysis, which contains triethyl citrate as an active ingredient either pure or in combination with some synergists. The triethyl citrate is in a quantity in weight expressed as a percentage from 0.1 to 99.9, preferably from 0.2 to 50 percent or even better a weight expressed as a percentage from 1.0 to 25.0. The claims are also directed to the use of triethylcitrate for the treatment of acne and seborrheic dermatitis, for the protection of triglycerides in alimentary products and for treatment of aesthetic cutaneous effects in the cosmic field.

### FIELD OF THE INVENTION

The present invention relates to a new composition for use in alimentary, cosmetic or pharmaceutical products for the protection of triglycerides from enzymatic hydrolysis.

### STATE OF THE ART

Triglycerides or triacylglycerols comprise three esterified fatty acids on a level with the three hydroxyl groups of glycerol. Triglycerides are hydrophobic and non-polar molecules.

Triglycerides form an important energy reserve both in simple and composite organisms, both of vegetable and animal origin.

Triglycerides are present in many foods such as, for indicative and not binding purposes, vegetable oils such as olive, sunflower, peanut oil etc, and in animal fats such as butter.

A particular functional role carried out by triglycerides on a skin level and specifically in precise pathological conditions such as acne, dermatitis seborrhoea and other pathologies in which the integrity of triglycerides becomes changed due to the action of certain enzymes (belonging to the esterase group and specifically to the lipase class) capable of hydrolysating the molecule, releasing fatty acids and glycerol.

According to the state of the art no chemical means exists that, when combined with triglycerides, is able to behave as a preferential substrata in regard to triglycerides for the enzymes which are part of the esterase group and more specifically of the lipase group (whether of bacterial, vegetable or animal origin).

Now, following specific research and experiments carried out by the inventor, it has emerged that an active ingredient, triethyl citrate, taken into consideration herein, performs a specific activity in inhibiting hydrolysis of triglycerides obtained enzymatically.

The inhibition of the lyses of triglycerides in the presence of porcine lipase was evaluated by analysing the released fatty acids using a colorimetric test.

The test consists in two distinct phases, one enzymatic reaction test which enables the lyses of triglycerides to leave the olive oil in the presence of porcine lipase and, successively, a colorimetric essay to determine the released fatty acids formed.

The porcine lipase, in the presence of the substrata (olive oil) and in opportune incubation conditions, catalyzes the following reaction:

Triglycerides+H2O→Diglycerides+Fatty acids

The dose of fatty acids allows indirect evaluation of the enzymatic activity and the lyses of triglycerides.

To evacuate the inhibition activity of the substance being analysed, three different concentrations of the substance under examination (5%, 1% and 0.5% in water) were tested at two different times (15 and 30 minutes).

In order to allow complete solubility, the 5% concentration was left at room temperature for 72 hours.

The porcine lipase was dissolved in deionised water at 4° C. just before use at the concentrations of 2000 U/ml. As a source of triglycerides olive oil was used. The buffer used for the reaction is Tris HCl 200 mM, pH=7.7 at 37° C. The test was conducted in the presence and in the absence of substance to be tested at different concentrations.

The following reagents are incubated at 37° C.: deionised water (150 μL), buffer (100 μL), olive oil (300 μL).

In the case of the sample (TEC 5%, 1% and 0.5%), the water was substituted with 150 μL of the solution to be tested.

Subsequent, the enzymatic solution (200 μL) was added and the reaction was allowed to develop for 30 and 15 minutes at a temperature of 37° C. On termination of the 15 and 30 minute periods, the reaction was blocked by adding 300 μL of ethanol at 95%.

50 μL of the solution containing the released fatty acids formed are used for the calorimetric dose, carried out using the special kit, that provides the necessary reagents to develop the reaction described below.

In the presence of the Acyl-CoA enzyme synthesis (AcylCS) and adenosin-5′-triphosphate (ATP) the released fatty acids are converted into acyl-CoA, adenosin-5′-monophosphate (AMP) and pyrophosphate. Acyl-CoA reacts with oxygen (O2) in the presence of Acyl coA oxidase (ACOD) forming 2,3-enoylcoenzymeA. The resulting H202 converts the 2,4,6-tribromo-3-hydroxy-benzoic acid (TBHB) and the 4-aminoantipyrine (4-AA) into a red stain in the presence of peroxidase (POD).

The samples are read at the 546 nm wave length, before and after the addition of ACOD and the reaction is conducted at room temperature.

Fatty acids+CoA+ATP→acyl-CoA+AMP+pyrophosphate

Acyl-CoA+O2→enoyl-coA+H202

H202+4-AA+TBHB→stain+2H2O+HBr

From the absorbance values it can be seen how triethyl citrate is able to protect the triglycerides from the enzymatic hydrolysis exercised by the lipases up to a percentage, according to the method followed, of up to 85%.

| | |
|---|---|
| | fatty acids |
| Absorbance | reduction compared |
| Mean | to control (%) |

| | 15′ | 30′ | 15′ | 30′ |
|---|---|---|---|---|
| Control | 0.7125 | 0.095 | — | — |
| TEC 0.5% | 0.584 | 0.084 | 18.04 | 11.10 |
| TEC 1% | 0.5905 | 0.083 | 17.12 | 12.70 |
| TEC 5% | 0.393 | 0.018 | 44.84 | 81.50 |

### OBJECTS OF THE INVENTION

This invention has been conceived on the basis of the results of this research, and therefore its primary objective is to propose the use of a new active principle useful at least in protecting the structure of the triglycerides in foods, in the organism and in a general sense in any case where it is useful and/or necessary to protect the molecular structure of triglycerides from enzymatic hydrolysis of triethyl citrate leads to the resulting release of citric acid, characterised by a marked antioxidant capacity capable of protecting molecular structures such as fatty acids from oxidisation and consequent molecular degradation.

The aim therefore of this invention is to provide an active principle for the protection of foods, for the formulation of products, both cosmetics and pharmaceutical, used either topically or by means of the systemic pathway (oral, intramuscular, intravenous, etc.) to obstruct and/or inhibit enzymatic hydrolysis of the triglycerides.

The invention has been found to be, among other things, particularly useful in some cutaneous pathologies such as seborrhoea, acne, seborrheic dermatitis and in a wider sense, any pathology characterised by the fact that hydrolysis of triglycerides and the successive releasing of fatty acids may lead to an aggravation of the clinical status or influence both directly and indirectly the etiopathogenesis of the illness.

A further aim of the invention is to provide the possibility to use different substances such as antioxidants, antibiotics, vitamins, retinoids, organic acids and in a wider sense other substances classified later as synergists whose action combined with triethyl citrate has resulted in being useful in avoiding degradation of foods and/or the control of certain pathologies and/or minor cutaneous defects.

According to the invention, these objectives are reached, by a composition for alimentary, cosmetic or pharmaceutical use, containing, as active ingredient, triethyl citrate in the pure form or in association with synergists.

The importance of maintaining the integrity of the triglycerides in acne is described indicatively even if not exhaustively.

Acne is a pathology that typifies the majority of male adolescents and which is characterised, in particular but not only, in the initial phase, by an increase in secretion of sebum (caused by the action of the male sex hormones).

Sebum is mainly made up of triglycerides which, under the action of bacterial lipases released by Propionibacterium acnes (a saprophyte bacterium which, as the illness progresses, increases in number releasing lipases) are hydrolysed accompanied by the releasing of fatty acids which, in their turn, under the action of free radicals and reactive species of the oxygen (SWR) released by the neutrophils in their turn recalled to destroy the excess Propionibacterium acnes, are degraded to smaller molecules ketones, aldehydes, etc.) characterised by a distinct inflammatory activity and accessory to the successive appearance of acne lesions.

The possibility of maintaining the integrity of the triglycerides is therefore closely related to a control, even if indirectly, of the progress of the inflammatory process and therefore of the typical cutaneous lesions such as papules, pustules, cysts and nodules.

### DETAILED DESCRIPTION OF THE INVENTION

In this invention and for the abovementioned use, triethyl citrate can be used pure with suitable supports or vehicles or, better, formulated with other chemical entities, such as synergists, additives and excipients, in a quantity in weight from 0.1 a 99.9%, preferably from 0.2 to 50%, better still from 1.0 to 25%, on the basis of the final formulation, for both alimentary, cosmetic and pharmaceutical preparations.

—Association with Appropriate Synergists—

In this invention, even if the individual use of triethyl citrate is found to have an exhaustive specific protective action on the triglycerides, the association with appropriate synergists can lead to an intensification of the result obtainable compared with a non-combined use of the different components.

Consequently, the active ingredient represented by triethyl citrate can be used, for example, in combination with substances which are part of the chemical group that comprises carboxylic acids, hydroxyacids, vitamins, amino acids, bioflavonoids, oligoelements, antibiotics, sulpha drugs, disinfectants, diethyl ethers of oleic, linoleic and linolenic acids and with other compositions such as for example, erythromycin, clindamycin, metronidazole, gentamycin, fusidic acid, econazole, ketoeconazole, mupirocin, hydrogen peroxide, benzoic peroxide, cetylpyridinium, silver and relative salts, whether organic or inorganic.

Synergists, for example, are intended to be as follows: trans retinoic acid, retinol, retinaldehyde, tocopherol, ascorbic acid, azelaic acid, octadecanediol acid, biotin, para aminobenzoic acid, rutina, β-carotene, thiamine, riboflavin, pyridoxine, pyridoxal, niacin, nicotinic acid, nicotinamid, pantothenic acid, panthenol, glucosamine, acetoglucosamine, folic acid, lecithin, phospholipids such as, for example, phosghatidylcholine, cephalin, phosphatide acid, lyso-phosphatidylcholine, hydroquinone, oleic acid, linoleic acid, linolenic acid, ethyl oleate, ethyl linolenate, ethyl lineolate, kojic acid, ascorbyl glucoside, erythromycin, clindamycin, metronidazole, gentamycin, fusidic acid, econazole, ketoeconazole, mupirocin, neomycin, streptomycin, hydrogen peroxide, benzoyl peroxide, cetylpyridinium, benzalkonium, Chlorhexidine and relative salts and esters, silver and relative salts, whether organic or inorganic hydroxyacids and beta hydroxyacids, both monocarboxylic and bicarboxylic, such as glycolic acid, lactic acid (in dextrose and levorotatory forms and in racemica mixture), hydroxybutyric acid (in dextrose and levorotatory forms and in racemica mixture), mandelic acid (in dextrose and levorotatory forms and in racemica mixture), tartaric acid (in dextrose and levorotatory forms and in racemica mixture malic acid (in dextrose and levorotatory forms and in racemica mixture), salicylic acid, 3-hydroxybenzoic acid, 4-hdroxybenzoic acid, cysteine, acetyl cysteine, glycine, selenium sulphur used individually or in association with two or more, including their respective salts, esters and amide and relative D-L-DL forms.

One or more components of this group of substances can be used in association with triethyl citrate in a quantity in weight expressed as a percentage from 0.01% to 50%, preferably from 0.5 to 15%.

The following examples of preparations are further evidence of the efficacy of the composition of this invention that contains triethyl citrate a san active ingredient.

Triethyl citrate, possibly associated with appropriate synergists as described above, can be used in formulations for external use, such as water-in oil emulsions, mono-phase solutions, biphasic pseudo-solution, mono-phase gels, biphasic gels, anhydrous ointments, aspersion powders, etc., using the supports or appropriate vehicles.

Examples of preparations with a triethyl citrate base to inhibit the alimentary oils to go rancid.

PREPARATION 1

| | | % in |
|---|---|---|
| N^{o} | Description | weight |
| 01 | Triethyl citrate | 0.1 |
| 02 | Olive oil for use with foodstuffs, as much as needed | 100 |

Method of preparation: use as is.

PREPARATION 2

| | | % in |
|---|---|---|
| N^{o} | Description | weight |
| 01 | Triethyl citrate | 0.1 |
| 02 | Sunflower oil for use with foodstuffs, as much as needed | 100 |

Method of preparation: use as is.

PREPARATION 3

| | | % in |
|---|---|---|
| N^{o} | Description | weight |
| 01 | Triethyl citrate | 0.1 |
| 02 | Peanut oil for use with foodstuffs, as much as needed | 100 |

Method of preparation: use as is.

Examples of preparations with a triethyl citrate base in the treatment of acne, of seborrheic dermatitis and all the pathologies in which an hydrolysis of triglycerides may lead to an aggravation of the clinical status/or may interfere with the etiopathogenesis of the illness and/or syndrome.

PREPARATION 4: Lotion for treating acne.

| | | % in |
|---|---|---|
| N^{o} | Description | weight |
| 01 | Triethyl citrate | 5 |
| 02 | Antibiotic* | 0.5-5 |
| 03 | Ethyl alcohol 95° | 60 |
| 04 | Distilled water, as much as needed | 100 |

Method of preparation: dissolve 02 in 03, add 04 to the solution obtained then mix in 01.
*antibiotic: compositions chosen for example between erythromycin, clindamycin, mupirocin, metronidazole, gentamycin.

PREPARATION 5: Lotion for the treatment of seborrheic
dermatitis

| | | % in |
|---|---|---|
| N^{o} | Description | weight |
| 01 | Triethyl citrate | 5 |
| 02 | antimycotics* | 0.5-5 |
| 03 | Ethyl alcohol 95° | 60 |
| 04 | Distilled water, as much as needed | 100 |

Method of preparation: dissolve 02 in 03, add 04 to the solution obtained then mix in 01.
*antimycotics: compositions chosen for example among fusidic, econazole, ketoeconazole acid

PREPARATION 6

| | | % in |
|---|---|---|
| N^{o} | Description | weight |
| 01 | Triethyl citrate | 0.3 |
| 02 | Sunflower oil for use with foodstuffs, as much as needed | 100 |

Method of preparation: use as it is

PREPARATION 7

| | | % in |
|---|---|---|
| N^{o} | Description | weight |
| 01 | Triethyl citrate | 0.3 |
| 02 | Peanut oil for use with foodstuffs, as much as needed | 100 |

Method of preparation: use as it is

PREPARATION 8

| | | % in |
|---|---|---|
| N^{o} | Description | weight |
| 01 | Triethyl citrate | 20.00 |
| 02 | Erythromycin | 2.00 |
| 03 | Ethyl alcohol | 60.00 |
| 04 | Demineralised Water | 18.00 |

Method of preparation: dissolve 02. in 03; mix 01 in the solution obtained; then add 04.

PREPARATION 9

| | | % in |
|---|---|---|
| N^{o} | Description | weight |
| 01 | Triethyl citrate | 6.00 |
| 02 | Salicylic Acid | 0.50 |
| 03 | Ethyl alcohol | 60.00 |
| 04 | Demineralised Water | 33.50 |

Method of preparation: dissolve 02. in 03; mix 01 in the solution obtained; then add 04.

PREPARATION 10

| | | % in |
|---|---|---|
| N^{o} | Description | weight |
| 01 | Triethyl citrate | 25.00 |
| 02 | Retinoic Acid | 0.025 |
| 03 | Ppg-15 stearyl ether, as much as needed | 100 |

Method of preparation: dissolve 02 in 03; disperse 01 in the solution obtained.

PREPARATION 11

| | | % in |
|---|---|---|
| N^{o} | Description | weight |
| 01 | Triethyl citrate | 95.00 |
| 02 | Ethyl linoleate | 5.00 |

Method of preparation: dissolve 02. in 01.

PREPARATION 12

| | | % in |
|---|---|---|
| N^{o} | Description | weight |
| | A) | |
| 01 | Triethyl citrate | 10.00 |
| 02 | Steareth-2 | 3.00 |
| 03 | Steareth-21 | 2.00 |
| 04 | Vaseline oil | 1.0 |
| 05 | Stearic Acid | 5.000 |
| | B) | |
| 06 | Preservatives | qb |
| 07 | Glycerol | 4.00 |
| 08 | Demineralised water, as much as needed | 100 |

Method of preparation: ingredients (A) and ingredients (B) are heated separately at 70° C. The ingredients (B) are added to ingredients (A) mixing until an accurately homogenised mixture in the form of an emulsion for topical use is reached.

PREPARATION 13

| | | % in |
|---|---|---|
| N^{o} | Description | weight |
| 01 | Triethyl citrate | 5.000 |
| 02 | Chlorhexidine gluconate | 0.250 |
| 03 | Hydroxyethyl cellulose | 1.000 |
| 04 | Demineralised water, as much as needed | 100 |

Method of preparation: dissolve 01. + 02. in 03. disperse 03 in the solution obtained, until complete solvation and formation of a gel has been reached.

1. A composition for topical use for the protection of triglycerides from enzymatic hydrolysis, comprising as an active ingredient pure triethyl citrate or triethyl citrate in combination with synergists.

2. A composition according to claim 1, which contains triethyl citrate in a quantity in weight expressed as a percentage from 0.1 to 99.9, preferably from 0.2 to 50 percent.

3. A composition according to claim 2, which contains triethyl citrate in a quantity in weight expressed as a percentage from 1.0 to 25.0 percent.

4. A composition according to claim 1, which contains the active ingredient represented by triethyl citrate in association with at least one of the additional substances chosen from between trans retinoic acid, retinol, retinaldehyde, tocopherol, ascorbic acid, azelaic acid, octadecanediol acid, biotin, para aminobenzoic acid, rutina, p-carotene, thiamine, riboflavin, pyridoxine, pyridoxal, niacin, nicotinic acid, nicotinamid, pantothenic acid, panthenol, glucosamine, acetoglucosamine, folic acid, lecithin, phospholipids such as, for example, phosghatidylcholine, cephalin, phosphatide acid, lysophosphatidylcholine, hydroquinone, oleic acid, linoleic acid, linolenic acid, ethyloleate, ethyl linolenate, ethyl lineolate, kojic acid, ascorbyl glucoside, erythromycin, clindamycin, metronidazole, gentamycin, fusidic acid, econazole, ketoeconazole, mupirocin, neomycin, streptomycin, hydrogen peroxide, benzoyl peroxide, cetylpyridinium, benzalkonium, Chlorhexidine and relative salts and esters, silver and relative salts, whether organic or inorganic hydroxyacids and beta hydroxyacids, both monocarboxylic and bicarboxylic, such as glycolic acid, lactic acid (in dextrose and levorotatory forms and in racemica mixture), hydroxybutyric acid (in dextrose and levorotatory forms and in racemica mixture), mandelic acid (in dextrose and levorotatory forms and in racemica mixture), tartaric acid (in dextrose and levorotatory forms and in racemica mixture malic acid (in dextrose and levorotatory forms and in racemica mixture), salicylic acid, 3-hydroxybenzoic acid, 4-hdroxybenzoic acid, cysteine, acetyl cysteine, glycine, selenium sulphur used individually or in association with two or more, including their respective salts, esters and amide and relative D-L-DL forms.

5. A composition according to claim 4, in which said additional substances are contained in a quantity in weight expressed as a percentage from 0.01% to 50% in weight, preferably from 0.5 to 15%.

6. A method comprising:
providing composition containing triethyl citrate;
using said composition as a substance for the protection of triglycerides from enzymatic hydrolysis in alimentary products.

7. A method comprising:
providing a composition containing triethyl citrate;
using said composition as a pharmaceutical substance for the treatment of pathologies, both directly and indirectly connected with hydrolysis of triglycerides.

8. A method comprising:
providing a composition containing triethyl citrate;
using said composition as a pharmaceutical substance for the treatment of acne and seborrheic dermatitis.

9. A method comprising:
providing a composition containing triethyl citrate;
using said composition as a cosmetic substance at least for the treatment of minor aesthetic cutaneous defects, both directly and indirectly connected to hydrolysis of triglycerides.

10. A composition according to claim 2, which contains the active ingredient represented by triethyl citrate in association with at least one of the additional substances chosen from between trans retinoic acid, retinol, retinaldehyde, tocopherol, ascorbic acid, azelaic acid, octadecanediol acid, biotin, para aminobenzoic acid, rutina, p-carotene, thiamine, riboflavin, pyridoxine, pyridoxal, niacin, nicotinic acid, nicotinamid, pantothenic acid, panthenol, glucosamine, acetoglucosamine, folic acid, lecithin, phospholipids such as, for example, phosghatidylcholine, cephalin, phosphatide acid, lysophosphatidylcholine, hydroquinone, oleic acid, linoleic acid, linolenic acid, ethyl oleate, ethyl linolenate, ethyl lineolate, kojic acid, ascorbyl glucoside, erythromycin, clindamycin, metronidazole, gentamycin, fusidic acid, econazole, ketoeconazole, mupirocin, neomycin, streptomycin, hydrogen peroxide, benzoyl peroxide, cetylpyridinium, benzalkonium, Chlorhexidine and relative salts and esters, silver and relative salts, whether organic or inorganic hydroxyacids and beta hydroxyacids, both monocarboxylic and bicarboxylic, such as glycolic acid, lactic acid (in dextrose and levorotatory forms and in racemica mixture), hydroxybutyric acid (in dextrose and levorotatory forms and in racemica mixture), mandelic acid (in dextrose and levorotatory forms and in racemica mixture), tartaric acid (in dextrose and levorotatory forms and in racemica mixture malic acid (in dextrose and levorotatory forms and in racemica mixture), salicylic acid, 3-hydroxybenzoic acid, 4-hdroxybenzoic acid, cysteine, acetyl cysteine, glycine, selenium sulphur used individually or in association with two or more, including their respective salts, esters and amide and relative D-L-DL forms.

11. A composition according to claim 3, which contains the active ingredient represented by triethyl citrate in association with at least one of the additional substances chosen from between trans retinoic acid, retinol, retinaldehyde, tocopherol, ascorbic acid, azelaic acid, octadecanediol acid, biotin, para aminobenzoic acid, rutina, p-carotene, thiamine, riboflavin, pyridoxine, pyridoxal, niacin, nicotinic acid, nicotinamid, pantothenic acid, panthenol, glucosamine, acetoglucosamine, folic acid, lecithin, phospholipids such as, for example, phosghatidylcholine, cephalin, phosphatide acid, lysophosphatidylcholine, hydroquinone, oleic acid, linoleic acid, linolenic acid, ethyl oleate, ethyl linolenate, ethyl lineolate, kojic acid, ascorbyl glucoside, erythromycin, clindamycin, metronidazole, gentamycin, fusidic acid, econazole, ketoeconazole, mupirocin, neomycin, streptomycin, hydrogen peroxide, benzoyl peroxide, cetylpyridinium, benzalkonium, Chlorhexidine and relative salts and esters, silver and relative salts, whether organic or inorganic hydroxyacids and beta hydroxyacids, both monocarboxylic and bicarboxylic, such as glycolic acid, lactic acid (in dextrose and levorotatory forms and in racemica mixture), hydroxybutyric acid (in dextrose and levorotatory forms and in racemica mixture), mandelic acid (in dextrose and levorotatory forms and in racemica mixture), tartaric acid (in dextrose and levorotatory forms and in racemica mixture malic acid (in dextrose and levorotatory forms and in racemica mixture), salicylic acid, 3-hydroxybenzoic acid, 4-hdroxybenzoic acid, cysteine, acetyl cysteine, glycine, selenium sulphur used individually or in association with two or more, including their respective salts, esters and amide and relative D-L-DL forms.
